(19) United States
(12) Patent Application Publication
(10) Pub. No.: US 2007/0122456 A1
(43) Pub. Date: May 31, 2007

(54) Nicotine and Cocoa Powder Compositions

(76) Inventor: Nils-Olof Lindberg, Malmo (SE)

Correspondence Address: FULBRIGHT & JAWORSKI, LLP, 1301 MCKINNEY, SUITE 5100, HOUSTON, TX, 77010-3095, US

(21) Appl. No.: 11/561,539

(22) Filed: Nov. 20, 2006

Related U.S. Application Data
(63) Continuation of application No. 10/271,186, filed on Oct. 15, 2002.
(60) Provisional application No. 60/329,369, filed on Oct. 15, 2001.

(51) Int. Cl. A61K 31/4439; A61K 47/00

(52) U.S. Cl. 424/439; 514/343

(57) ABSTRACT

The present invention relates to a nicotine-containing pharmaceutical composition and methods of using the composition in therapies, such as nicotine replacement therapy.

### CROSS-REFERENCE TO RELATED APPLICATIONS

This application is a continuation of U.S. application Ser. No. 10/271,186 filed Oct. 15, 2002 which claims priority to U.S. Provisional Application No. 60/329,369, which was filed on Oct. 15, 2001, each of which is incorporated herein by reference.

### TECHNICAL FIELD

This invention relates to novel pharmaceutical compositions of nicotine and use thereof. More particularly, the present invention relates to compositions comprising nicotine and cocoa powder, methods to prepare the compositions, and to methods for using the compositions in nicotine replacement therapy (NRT), including tobacco substitution and smoking cessation.

### BACKGROUND OF THE INVENTION

Nicotine replacement therapy as a smoking cessation strategy has been successful in the past. Previous nicotine-containing compositions aiming towards the purpose of reducing nicotine craving for subjects wishing to stop their use of tobacco products include e.g., U.S. Pat. No. 3,845,217 disclosing chewable compositions, U.S. Pat. No. 4,579,858 disclosing high-viscous nicotine nose-drop compositions, U.S. Pat. No. 5,525,351 disclosing nicotine-containing saliva-soluble gels, U.S. Pat. No. 5,656,255 disclosing low-viscous nicotine-containing compositions suitable for nasal spray administration, U.S. Pat. No. 4,920,989, U.S. Pat. No. 4,953,572 and U.S. Pat. No. 5,167,242 disclosing the use of inhalation aerosol, BP 1,528,391 and BP 2,030,862 disclosing liquid aerosol formulations adapted as mouth-sprays, and devices for transdermal delivery of nicotine.

A well-known side effect of nicotine is related to its concentration dependent local irritation. This adverse effect is particularly noticeable when nicotine formulations are applied topically, including transmucosal routes, comprising buccal and nasal, and transdermal administration routes.

UK Patent application GB 2230439A describes nicotine lozenges with a shell or coating containing an oral-acting local analgesic, preferably eugenol. Though not stated explicitly to be the cause of the so included local analgesic, the aforesaid disclosure is said to substantially ameliorate the sensation of burning in the mouth experienced with conventional nicotine lozenges. Similarly, nicotine-compositions formulated in lozenges containing local analgesic have been disclosed in AU 662877 in which the latter agent is said to temporarily interfere with taste receptors which is said to reduce the de-sire to eat.

The concentration of nicotine in several of the above-mentioned inventions, and product designs thereof, is hence limited by adverse effects caused by or related to its local irritation.

Prior art describes other capsules, tablets, and lozenges for oral delivery of nicotine. For example, WO 88/03803 discloses a chewable capsule filled with a liquid containing 0.1-10.0 mg of nicotine, together with additives for improving flavor and dispersion. The capsules are provided in a variety of pH values to allow the patient a choice of nicotine absorption rates, and are especially intended as an aid to quit smoking.

Another nicotine capsule formulation is disclosed by Jarvik et al. (Clinical Pharmacology and Therapeutics 1970; 11: 574) for ingestion as a smoking cessation aid. The subjects, according to the theory that intestinal absorption of nicotine could produce significant blood levels, however, apparently swallowed these capsules whole. The study showed a small but significant decrease in the number of cigarettes smoked by subjects, but no quantitative measurements of nicotine blood levels were obtained.

BE 899037 discloses a tablet containing 0.1 to 5 mg nicotine as a base or water-soluble acid salt as an aid for quitting smoking.

Shaw (for example in GB 2142822 and U.S. Pat. No. 4,806,356) describes a nicotine lozenge prepared from a mixture of inert filler material, a binder, and either pure nicotine or a nicotine-containing substance by cold compression.

U.S. Pat. No. 5,512,306 discloses a nicotine product for oral delivery in the form of an inclusion complex of nicotine and a cyclodextrin compound. It also discusses the use of various excipients and direct compression for manufacture of the product.

WO 97/42941 discloses a slowly erodible nicotine lozenge that allows delivery to the buccal mucosa over an extended period of time.

U.S. Pat. No. 5,662,920 discloses a nicotine lozenge that may contain candy taste flavorants, such as chocolate, orange, vanilla, as well as other flavorants. No amount sufficient for taste-masking is though suggested. Further, cocoa powder is not disclosed.

The literature also describes different designs of tablets for delivering nicotine to the mouth and digestive system.

Wesnes and Warburton (Psychopharmacology 1984; 82:147; Psychopharmacology 1986; 89:55) discuss the use of nicotine-containing dextrose and magnesium hydroxide tablets. The subjects were instructed to keep the tablets in the mouth for some minutes before swallowing, in order to maximize contact with the buccal mucosa.

Several products based on the above mentioned patents are now marketed on an international scale. In addition, several nicotine lozenges are available as over-the-counter products in the UK Resolution lozenges, manufactured by Phoenix Pharmaceuticals and distributed by Ernest Jackson, which contain 0.5 mg nicotine, together with the anti-oxidant vitamins A, C and E. Stoppers lozenges, distributed by Charwell Pharmaceuticals Ltd., contain 0.5 mg nicotine and are available in chocolate, orange and pepper-mint flavors.

There are, however, subjects who may have cravings for higher doses of nicotine than those acceptable in applications of prior art and subjects that may not experience a decrease in other withdrawal symptoms because of unsatisfactory nicotine absorption. Furthermore, it has to date been difficult to deliver nicotine in a profile mimicking the nicotine blood levels achieved by consistent smoking, to satisfy cravings for nicotine in people who are attempting to quit smoking, and thus, to provide greater protection against relapse than nicotine replacement therapies is possible with hitherto known. Thus, absorption of nicotine in the use of currently marketed products and as disclosed in prior art of nicotine replacement therapies does not satisfactorily resemble the use of tobacco products, in particular smoking. With chewing gum nicotine replacement therapy for smoking cessation blood peak levels of nicotine is reached after 30 minutes with venous blood nicotine levels about ⅓ to ⅔ of the levels attained when smoking (British Medical Journal 1976;1:1043). A smoker will usually reach peak blood levels of nicotine 5-10 minutes after starting smoking. It is therefore desirable to provide improved compositions and methods which avoid the disadvantages of these conventional nicotine delivery de-vices and methods while providing an effective means for delivering nicotine for smoking cessation treatment, for reducing nicotine craving, and for treating other conditions responsive to nicotine therapy.

An attempt to solve the captioned problems is made with a nicotine-containing composition, preferably for buccal uptake, according to WO 00/30641. Herein is disclosed a composition comprising nicotine, at least one apolar component, at least one polar component and at least one surface-active component. Many apolar components are suggested, including lipids such as cocoa butter and cocoa butter alternatives, including cocoa butter equivalents (CBE), cocoa butter substitutes (CBS), cocoa butter replacers (CBR) and cocoa butter improvers (CBI). Anyhow, the composition according to WO 00/30641 has the disadvantage of insufficient taste-masking of nicotine and buffering agents, and the drawback of causing nausea with some users. Cocoa powder is mentioned in one example, where the percentage though is so low that the cocoa powder may serve only as flavorant, not as taste-masking agent.

A few patent applications disclose cocoa powder as an excipient in different formulations, for example WO 00/51570 disclosing a drug-containing soft capsule comprising cocoa powder, primarily intended for swallowing and drug uptake in the stomach. JP 200095710 discloses compacted tablets comprising cocoa powder and vitamins or iron compounds. WO 00/13523 discloses an encapsulated matrix composition comprising caffeine and a fairly low percentage of cocoa powder. ES 21059710 and WO 95/24890 disclose formulations with certain antibiotics and cocoa powder. JP 93010326 discloses an oily formulation wherein cocoa powder per se is the active ingredient.

Chocolate, which is very different from cocoa powder as such, is very rarely used as an ingredient in pharmaceutical products, hitherto only in laxatives. One example is Ex-Lax® being chocolated laxative pieces marketed by Novartis comprising sennosides. In the 1950s, a laxative was marketed Purex having phenolphthalein that was formulated with chocolate. The Stoppers lozenges mentioned above do not comprise chocolate, or cocoa, but only chocolate flavors. Such chocolate flavors are not useful for the objectives of the present invention.

The present invention has found that a rapid buccal absorption of nicotine concomitantly with sufficient soothing of the burning sensation of nicotine and sufficient taste-masking of badly tasting ingredients, such as buffering agents, is achieved through the use of nicotine-containing formulations comprising cocoa powder as a vehicle or taste-masking agent, also serving as filler/diluent and smoothening/flavoring agent. No similar formulations have been disclosed hitherto. Thus, the present application is the first to use cocoa powder as a taste-masking agent for nicotine.

### BRIEF SUMMARY OF THE INVENTION

The present invention is to compositions for the therapeutic delivery of nicotine are provided. The compositions comprising nicotine provide rapid transmucosal absorption of nicotine. The compositions are preferably used for therapeutic administration of nicotine. Yet further, the pharmaceutical compositions of nicotine are formulated for uptake buccally or by other mucosa in the oral cavity.

An embodiment of the present invention is a nicotine-containing pharmaceutical composition comprising cocoa powder. Preferably, the amount of cocoa powder contained in the composition is at least a taste-masking effective amount of cocoa powder. More preferably, the cocoa powder is also the diluent agent, filler agent, smoothening agent, and flavoring agent. Yet further, the nicotine-containing pharmaceutical may comprise at least one compound selected from the group consisting of sucrose, fructose, glucose, galactose, lactose, maltose, invert sugar, xylitol, sorbitol, maltitol, mannitol, isomalt, glycerol, polydextrose, and any mixture thereof.

A further embodiment is the nicotine-containing pharmaceutical composition further comprising one or more lipid components. Preferably, the lipid is cocoa butter or cocoa butter alternatives. Exemplary cocoa butter alternatives, include, but are not limited to cocoa butter equivalents (CBE), cocoa butter substitutes (CBS), cocoa butter replacers (CBR) and cocoa butter improvers (CBI). Yet further, the lipid can be selected from the group consisting of oils based on lauric and myristic acids (i.e., coconut oil or palmkernel oil), oils based on palmitic, oleic and stearic acids (i.e., palm oil, shea butter, karite butter, illipe butter, mango kernel oil, and sal fat), oils based on oleic, linoleic and linolenic acids (i.e., corn oil, sunflower oil, hybrid sunflower oil, soybean oil, rapeseed oil, canola oil, olive oil, rice bran oil, cottonseed oil, peanut oil and groundnut oil) and oils based on animal fat (i.e., fish oil, tallow, lard, or butterfat). It is also envisioned that the lipid can be a synthetic fat, reesterified fat, or hard fat.

Still further, the nicotine-containing pharmaceutical composition comprises a buffering agent. Exemplary buffering agents include, but are not limited to sodium carbonates, sodium bicarbonates, sodium phosphates, sodium glycinates, sodium acetates, sodium gluconates, sodium glycerophosphates, potassium carbonates, potassium bicarbonates, potassium phosphates, potassium glycinates, potassium acetates, potassium gluconates, potassium glycerophosphates, ammonium carbonates, ammonium bicarbonates, ammonium phosphates, ammonium glycinates, ammonium acetates, ammonium gluconates, ammonium glycerophosphates and mixtures thereof. Preferably, the buffering agent is sodium carbonate.

Yet, still further, the nicotine-containing pharmaceutical composition comprises at least one emulsifier/solubiliser, for example, lecithin (i.e., soy lecithin or egg lecithin), a nonionic surfactant (i.e., poloxamer, polyoxyethylene alkyl ether, polyoxyethylene castor oil derivative, polyoxyethylene sorbitan fatty acid ester, monoglyceride, diglyceride, polyoxyethylene stearate, polyglycerolester of fatty acids and sorbitan fatty acid ester), an anionic surfactant (i.e., fatty acid, soap of fatty acid, lactylate, sodium lauryl sulfate and latanol), a zwitterionic surfactant (i.e., zwitterionic phospholipid ) and combinations with lecithin. Sweeteners can also be included, for example, aspartame, acesulfame potassium, saccharine, cyclamate, glycyrrhizine, dihydrochalcones, stevisoide, thaumatin, monellin and neohesperidine. Yet further, a flavoring agent can also be included, for example, peppermint, coffee, orange and vanilla.

A specific embodiment of the present invention is a nicotine-containing pharmaceutical composition having a unit dose which comprises from about 0.5 mg to about 10 mg of nicotine, from about 17% to about 70% (w/w) of cocoa powder, from about 20% to about 50% (w/w) of a lipid component, from about 0.3% to about 3% (w/w) of a sweetener, from about 0% to about 10% (w/w) of a buffering agent, from about 0.3% to about 5% (w/w) of a emulsifier/solubilizer and from 0% to about 4% (w/w) of a flavoring agent.

Another specific embodiment is a nicotine-containing pharmaceutical composition having a unit dose which comprises from about 1 mg to about 6 mg of nicotine, 50% (w/w) cocoa powder, 44% (w/w) lipid components, 15 mg sodium carbonate, 0.6% (w/w) aspartame and/or acesulfame potassium and about 1% (w/w) lecithin.

Another embodiment is a method for nicotine replacement therapy (NRT) comprising the step of administering to a subject in need of such therapy a unit dose of a nicotine-containing pharmaceutical composition, wherein the unit dose of the composition comprises from about 0.5 mg to about 10 mg of nicotine, from about 17% to about 70% (w/w) cocoa powder, from about 20% to about 50% (w/w) a lipid component, from about 0% to about 10% (w/w) of a buffering agent, from about 0.3% to about 3% (w/w) of a sweetener and from about 0.3% to about 5% of an emulsifier/solubilizer. Preferably, administering is via an oral route. Yet further, a second formulation of nicotine is also administered to the subject. The second formulation is administered via a device for transdermal administration of nicotine or is administered nasally or buccally or is administered via inhalation.

Yet further, another embodiment is a method of treating a subject suffering from nicotine addiction comprising administering to the subject the nicotine-containing composition of the present invention. Yet further, the composition may also be administered to a subject suffering from Alzheimer's disease, Parkinson's disease, Tourette's syndrome or ulcerative colitis. In further embodiments, the composition is also administered to a subject suffering from obesity. It is envisioned that the composition may also be used to control the weight of a subject.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description and is not intended as a definition of the limits of the present invention.

### DETAILED DESCRIPTION OF THE INVENTION

### A. DEFINITIONS

As used herein, the use of the word “a” or “an” when used in conjunction with the term “comprising” in the sentences and/or the specification may mean “one,” but it is also consistent with the meaning of “one or more,” “at least one,” and “one or more than one.”

In the absence of explicit statements to the contrary, as used herein expressions like “comprising”, “including”, “having”, “with” and similar terminology shall not be understood to be exclusively restricted to the recited element(s), but shall be understood to allow for the presence of further elements as well, and shall be understood to cover any element(s) in integral, subdivided or aggregate forms, as well to imply the inclusion of a stated integer or step or group of integers or steps, but not the exclusion of any other integer or step or group of integers or steps.

The term “buccal” as used herein is defined as for uptake buccally or by other mucosa in the oral cavity.

The term “disintegration” as used herein denotes melting, solubilization, erosion or a combinatorial effect of these physical changes of the invention.

The term “oral administration” as used herein includes oral, buccal, enteral or intragastric administration.

The term “transmucosal administration” or “transmucosal delivery” as used herein means any system or device for the administration of a drug across a subject's mucosal membrane, including the oral mucosa, such as the buccal and sublingual mucosa, and other mucosal membranes, including rectal, nasal, and vaginal. See “Controlled Drug Delivery, Fundamentals and Applications”, 2nd Ed., Robinson and Lee, eds., Chapter 1, “Influence of Drug Properties and Routes of Drug Administration on the Design of Sustained and Controlled Release Systems”, Li et al., Marcel Dekker Inc.: New York, pp. 3-61 (1987).

The term “subject” as used herein, is taken to mean any mammalian subject to which a nicotine-containing composition is orally administered according to the methods described herein. In a specific embodiment, the methods of the present invention are employed to treat a human subject. Another embodiment includes treating a human subject in need of nicotine replacement therapy.

The term “taste-masking agent” used herein refers to an agent that is added to a composition to mask the taste of badly tasting components in the composition. For example, cocoa powder in the present invention masks the taste of nicotine. Yet further, as used herein the terms “taste-masking agent” and “vehicle” are interchangeable.

The term “therapeutically effective amount” as used herein refers to an amount that results in an improvement or remediation of the symptoms of the disease or condition.

The term “treating” and “treatment” as used herein refers to administering to a subject an effective amount of a nicotine-containing composition so that the subject has an improvement in the disease or condition. The improvement is any improvement or remediation of the symptoms. The improvement is an observable or measurable improvement. Thus, one of skill in the art realizes that a treatment may improve the disease or condition, but may not be a complete cure for the disease or condition.

The term “prophylactic” as used herein is defined as a drug or agent which acts to prevent a disease or condition, e.g., a vaccine.

### B. PHARMACEUTICAL COMPOSITIONS

It is an object of the present invention to provide a nicotine-containing pharmaceutical composition. More specifically, it is the object of the invention to provide such a nicotine-containing composition for transmucosal, preferably buccal, delivery, which disintegrates and/or melts at body temperature with or without the aid of salivary fluid or mechanical erosion, or a combination thereof after which the formulation preferably shows adhesiveness towards the tissues in the oral cavity. This form of drug delivery provides for an efficient entry of active substances to the systemic circulation and reduces immediate metabolism by the liver and intestinal wall flora.

In preferred embodiments, the active ingredient of the composition is nicotine and cocoa powder is the vehicle for the active ingredient. The composition may comprise further components for example, but not limited to fatty and/or lipid components, sweeteners, flavoring agents, buffering agents, emulsifers/solubilzers or other components.

The nicotine may be present in any suitable form, i.e., as free base, as a salt or as a complex. There is no need to use nicotine in a microencapsulated form. The free base is extremely volatile and is absorbed readily through mucous membranes and intact skin. The major problems reported for products based on nicotine free base originate from the volatility of the nicotine, its acrid, burning taste, the irritating sensation on the mucous membranes, and the decomposition of nicotine in the presence of oxygen. Previously, these problems have been alleviated, in part, through the use of nicotine's salt form, i.e., an acid addition salt or metal salt. The present invention utilizes cocoa powder as a vehicle to counter some of the problems associated with using a free base of nicotine, for example, burning taste. It is also envisioned that cocoa powder, in addition to acting as a taste-masking agent, may also serve as a smoothening and flavoring agent and/or as a filler and diluent agent. Thus, as used herein, cocoa powder masks the taste of nicotine and/or other badly tasting components such as buffering agents.

Cocoa nib is defined as cocoa beans with the shell removed. Cocoa mass is defined as cocoa nib ground to give a substance being a liquid above 35° C. Cocoa liquor is another name for cocoa mass. Cocoa powder is defined as cocoa nib with some fat removed and ground into a powder. Cocoa butter is defined as fat expelled from the center (kernels or nib) of cocoa beans.

Cocoa powder is prepared from roasted cocoa beans. It is a complex compound, which consists of starch, cocoa butter, amino acids, proteins, xanthines, amines, mono- and polysaccharides, phospholipids, flavonoids, pyrazines, etc.

It is important to note that there is an essential difference between chocolate and cocoa powder as such. According to Industrial Chocolate Manufacture and Use, S. T. Beckett, ed., 2nd edition, Blackier Academic & Professional, London, 1994, p 382, chocolate is defined as a product obtained from cocoa nib, cocoa mass powder and sucrose with or without added cocoa butter, having a minimum dry cocoa solids content of 35%, at least 14% of dry non-fat cocoa solids and 18% cocoa butter. Chocolate has two major distinguishing characteristics: its flavor and its texture. A primary feature of the texture is that the chocolate must be solid at a temperature of 20-25° C. and yet melt rapidly in the mouth at 37° C. thereby being transferred to a liquid, which appears smooth to the tongue. The processing of chocolate is related to obtaining these two criteria (Beckett, p 2). Chocolate as such according to the definition above is not suitable in the formulation according to the present invention.

Neither milk chocolate nor light cooking chocolate or dark cooking chocolate may mask the disagreeable taste of most buffering agents. The cocoa content of milk chocolate is comparatively low (a cocoa mass content of 10-16%, corresponding to approximately 5-8% cocoa powder). The beans'/cocoa mass' content of dark, bitter-sweet chocolate is 55-70% (Beckett, pp. 276-277), corresponding to approximately 28-35% cocoa powder. By making a vehicle with a high proportion of cocoa powder (30-50%) and fatty components (40-45%), as per the present invention, an effective taste-masking is though obtained. The higher the cocoa powder concentration the better the taste-masking. Thus, an effective taste-masking amount is an amount of cocoa powder that results in masking the taste of nicotine.

Other useful embodiments of the present invention are obtained by exchanging some of the above-mentioned excipients for equivalently functioning alternative compounds. For example, a small part of the cocoa powder, acting as diluent/filler and taste-masking/smoothening/flavoring agent, may be exchanged for one or more of the compounds sucrose, fructose, glucose, galactose, lactose, maltose, invert sugar, a pharmaceutically acceptable polyol such as xylitol, sorbitol, maltitol, mannitol, isomalt and glycerol, or polydextrose, or any mixture thereof, but only to such an extent that the taste-masking effect of the cocoa-powder remains sufficient.

The lipid ingredient of the present invention, being fatty components, may be chosen from one or more of the following compounds: cocoa butter and cocoa butter alternatives (i.e., cocoa butter equivalents (CBE), cocoa butter substitutes (CBS), cocoa butter replacers (CBR) and cocoa butter improvers (CBI)); coconut, palmkernel oil and other similar oils (other similar oils include oils that are characterized by being predominantly based on lauric and myristic acids; palm oil, shea butter, karite butter, illipe butter, mango kernel oil, sal fat and other similar fats (other similar fats include fats that are characterized by being predominantly based on palmitic, oleic and stearic acids); corn oil, sunflower oil, hybrid sunflower oil, soybean oil, rapeseed oil, canola oil, olive oil, rice bran oil, cottonseed oil, arachis (peanut, groundnut) oil and other oils (other oils include oils that are characterized by being predominantly based on oleic, linoleic and linolenic acids and hydrogenated to a suitable melting point); fish oil, tallow, lard, butterfat and other animal derived fats; and synthetic fats, reesterified fats, hard fats obtained by a chemical reaction of fatty acids with glycerol using no, acidic, alkaline or enzymatic catalysis. The compounds can be used as a single component or mixed with each other, being either crude or refined using physical or alkaline refining, or being subjected to further processing including catalytic hydrogenation, interesterification, transesterification and fractionation. Preferred fatty components are fats/lipids chosen from tempering fats, including cocoa butter equivalents (CBE) and cocoa butter improvers (CBI), and non-tempering fats, including cocoa butter replacers (CBR) and cocoa butter substitutes (CBS).

The buffer sodium carbonate may be exchanged for carbonates, bicarbonates, acetates, gluconates, glycerophosphates, phosphates, glycinates, citrates, malates and/or tartrates of sodium, potassium or ammonium, or mixtures thereof. Most phosphates are though less suitable because their taste usually is disagreeable and difficult to mask.

The sweetener aspartame may entirely or in part be exchanged for one or more other artificial sweeteners, such as acesulfame potassium, saccharine, cyclamate, glycyrrhizine, dihydrochalcones, stevioside, thaumatin, monellin and/or neohesperidine.

The emulsifier lecithin is preferably soy lecithin and/or egg lecithin, but may be exchanged for a nonionic surfactant (i.e., poloxamer, polyoxyethylene alkyl ether, polyoxyethylene castor oil derivative, polyoxyethylene sorbitan fatty acid ester, mono-glyceride, diglyceride and esther thereof, polyoxyethylene stearate, polyglycerolester of fatty acids (including polyglycerolpolyricinoleic acid (PGPR)), sorbitan fatty acid ester); an anionic surfactant (i.e., fatty acid, soap of fatty acid, lactylate, especially sodium and/or calcium stearoyllactylate, sodium lauryl sulfate and latanol); a zwitterionic surfactant (i.e., zwitterionic phospholipid, such as phosphati-dylcholine and phosphatidylethanolamine) or mixtures, fractions or derivatives thereof or with lecithin.

If cocoa mass comprising phospholipids is used instead of part of the cocoa powder the emulsifier/solubilizer may be removed from the composition.

Optionally, liquid or solid flavoring agents may be added. Non-limiting examples of flavoring agents are peppermint, coffee, orange and vanilla.

The preferred formulation is a tablet melting in the mouth, weighing around 400 mg. Preferably, the tablet comprises nicotine. In preferred embodiments, it is envisioned that the concentration ranges for the respective components of the formulation per unit dose are as follows: from about 0.5 mg to about 10 mg of nicotine, from about 17% to about 70% (w/w) of diluent/filler agent, from about 17% to about 70% (w/w) of taste-masking agent, from about 17% to about 70% (w/w) of smoothening agent, from about 17% to about 70% (w/w) of flavoring agent, from about 20% to about 50% (w/w) of lipid component, from about 0.3% to about 3% (w/w) of sweetener, from about 0% to about 10% (w/w) of buffering agent, from about 0.35 to about 5% (w/w) of emulsifer/solubilizer, and from about 0% to about 4% (w/w) of flavoring agent. More preferably, the formulation comprises 1-6 mg of nicotine (as base or hydrogen tartrate); about 50% cocoa powder, about 44% fatty/lipid components, about 0.6% aspartame, about 15 mg of sodium carbonate, and about 1% lecithin. The percentages are w/w. Still further, other oral drug dosage forms may also include, lozenges, capsules, or gum. The methods of manufacture of these formulations are known in the art, for example, as described in U.S. Pat. No. 4,806,356, which is incorporated herein by reference.

Upon formulation, solutions are administered in a manner compatible with the dosage formulation and in such amount as is therapeutically effective to result in an improvement or remediation of the symptoms. The formulations are easily administered in a variety of dosage forms such as ingestible tablets and the like. Some variation in dosage can occur depending on the condition of the subject being treated. The person responsible for administration can, in any event, determine the appropriate dose for the individual subject. Moreover, for human administration, preparations meet sterility, general safety and purity standards as required by FDA Office of Biologics standards.

### C. NICOTINE THERAPY REPLACEMENT

It is the primary object of the present invention to provide a tobacco supplement or a tobacco substitute, for use in e.g. smoking cessation and nicotine replacement therapies, which provide the user with a satisfactory dose of nicotine, which is contained in the nicotine-containing composition of the present invention, so as to reduce tobacco withdrawal symptoms without causing unacceptable adverse effects. Yet further, it is envisioned that the addition of the taste-masking agent, cocoa powder, will reduce and/or eliminate the bad taste of the nicotine and/or other badly tasting components, such as buffering agents. Thus, the nicotine-containing composition of the present invention will be more desirable to the user.

A specific embodiment of the present invention is a method for nicotine replacement therapy (NRT) comprising the step of administering to a subject in need of such therapy a unit dose of a nicotine-containing pharmaceutical composition, wherein the unit dose of the composition comprises from about 0.5 mg to about 10 mg of nicotine, from about 17% to about 70% (w/w) cocoa powder, from about 20% to about 50% (w/w) a lipid component, from about 0% to about 10% (w/w) of a buffering agent, from about 0.3% to about 3% (w/w) of a sweetener and from about 0.3% to about 5% of an emulsifier/solubilizer. Preferably, administering is via an oral route.

In further embodiments, the nicotine-containing composition of the present invention may be administered in combination with a second formulation for nicotine replacement therapy. This second formulation may be a device for transdermal administration of nicotine, a spray for nasal, buccal or pulmonary uptake, a chewing gum, or a dosage form for oral or peroral use or any device for administration of tobacco.

### D. NICOTINE THERAPY FOR CENTRAL NERVOUS SYSTEM DISORDERS

Another aspect to the present invention is a method for the prevention and treatment of a central nervous system (CNS) disorder (i.e., Alzheimer's disease, Parkinson's disease, or Tourette's syndrome) by administering the nicotine-containing composition to a subject susceptible to or suffering from such a disorder.

CNS disorders are a type of neurological disorder. CNS disorders can be drug induced; can be attributed to genetic predisposition, infection or trauma; or can be of unknown etiology. CNS disorders comprise neuropsychiatric disorders, neurological diseases and mental illnesses; and include neurodegenerative diseases, behavioral disorders, cognitive disorders and cognitive affective disorders. There are several CNS disorders whose clinical manifestations have been attributed to CNS dysfunction (i.e., disorders resulting from inappropriate levels of neurotransmitter release, inappropriate properties of neurotransmitter receptors, and/or inappropriate interaction between neurotransmitters and neurotransmitter receptors). Several CNS disorders can be attributed to a cholinergic deficiency, a dopaminergic deficiency, an adrenergic deficiency and/or a serotonergic deficiency. CNS disorders of relatively common occurrence include presenile dementia (early onset Alzheimer's disease), senile dementia (dementia of the Alzheimer's type), Parkinsonism including Parkinson's disease, Huntington's chorea, tardive dyskinesia, hyperkinesia, mania, attention deficit disorder, anxiety, dyslexia, schizophrenia and Tourette's syndrome.

It is known that nicotine has certain pharmacological effects, for example neurotransmitter release. Exemplary neurotransmitters that are released upon administration of nicotine include but are not limited to acetylcholine, dopamine (Rowell et al., J. Neurochem., Vol. 43, pp. 1593-1598 (1984); Rapier et al., J. Neurochem., Vol. 50, pp. 1123-1130 (1988); Sandor et al., Brain Res., Vol. 567, pp. 313-316 (1991)), norepinephrine (Hall et al., Biochem. Pharmacol., Vol. 21, pp. 1829-1838 (1972)), serotonin (Hery et al., Arch. Int. Pharmacodyn. Ther., Vol. 296, pp. 91-97 (1997)), and glutamate (Toth et al., Neurochem Res., Vol. 17, pp. 265-271 (1992)). Therefore, it is desirable to provide to a subject susceptible to or suffering from a CNS disorder a pharmaceutical composition containing nicotine, which elicits neurotransmitter release within the subject in order to prevent or treat a neurological disorder. In addition, the nicotine-containing composition of the present invention may also potentiate the pharmacological behavior of certain pharmaceutical compositions typically used for the treatment of certain CNS disorders. See, Sanberg et al., Pharmacol. Biochem. & Behavior, Vol. 46, pp. 303-307 (1993); Harsing et al., J. Neurochem., Vol. 59, pp. 48-54 (1993) and Hughes, Proceedings from Intl. Symp. Nic., S40 (1994). Thus, the nicotine-containing composition of the present invention can be used alone or in combination with other standard CNS therapies.

1. Alzheimer's Disease

Senile dementia of the Alzheimer's type (SDAT) is a debilitating neurodegenerative disease, mainly afflicting the elderly; characterized by a progressive intellectual and personality decline, as well as a loss of memory, perception, reasoning, orientation and judgment. One feature of the disease is an observed decline in the function of cholinergic systems, and specifically, a severe depletion of cholinergic neurons (i.e., neurons that release acetylcholine, which is believed to be a neurotransmitter involved in learning and memory mechanisms). See, Jones, et al., Intern. J. Neurosci., Vol. 50, p. 147 (1990); Perry, Br. Med. Bull., Vol. 42, p. 63 (1986) and Sitaram, et al., Science, Vol. 201, p. 274 (1978). It has been observed that nicotinic acetylcholine receptors, which bind nicotine and other nicotinic agonists with high affinity, are depleted during the progression of SDAT. See, Giacobini, J. Neurosci. Res., Vol. 27, p. 548 (1990); and Baron, Neurology, Vol. 36, p. 1490 (1986).

In certain embodiments, it is envisioned that administering the nicotine-containing composition of the present invention to a subject suffering form SDAT can ameliorate some symptoms of SDAT. It is contemplated that acute administration of the composition will activate nicotinic cholinergic receptors, and chronic administration of the composition will elicit an increase in the number of such receptors. See, Rowell, Adv. Behav. Biol., Vol. 31, p. 191 (1987); and Marks, J. Pharmacol. Exp. Ther., Vol. 226, p. 817 (1983).

2. Parkinson's Disease

Parkinson's disease (PD) is a debilitating neurodegenerative disease, presently of unknown etiology, characterized by tremors and muscular rigidity. A feature of the disease appears to involve the degenerative of dopaminergic neurons (i.e., which secrete dopamine). One symptom of the disease has been observed to be a concomitant loss of nicotinic receptors which are associated with such dopaminergic neurons, and which are believed to modulate the process of dopamine secretion. See, Rinne, et al., Brain Res., Vol. 54, pp. 167-170 (1991) and Clark, et al., Br. J. Pharm., Vol. 85, pp. 827-835 (1985).

In certain embodiments, it is envisioned that administering the nicotine-containing composition of the present invention to a subject suffering form PD may ameliorate symptoms of PD.

3. Tourette's Syndrome

Tourette's syndrome (TS) is an autosomal dominant neuropsychiatric disorder characterized by a range of neurological and behavioral symptoms. Typical symptoms include (i) the onset of the disorder before the age of 21 years, (ii) multiple motor and phonic tics although not necessarily concurrently, (iii) variance in the clinical phenomenology of the tics, and (iv) occurrence of quasi daily tics throughout a period of time exceeding a year. Motor tics generally include eye blinking, head jerking, shoulder shrugging and facial grimacing; while phonic or vocal tics include throat clearing, sniffling, yelping, tongue clicking and uttering words out of context. The pathophysiology of TS presently is unknown, however it is believed that neurotransmission dysfunction is implicated with the disorder. See, Calderon-Gonzalez et al., Intern. Pediat., Vol. 8(2), pp. 176-188 (1993) and Oxford Textbook of Medicine, Eds. Weatherall et al., Chapter 21.218 (1987).

A further embodiment of the present invention comprises administering to a subject suffering from TS the nicotine-containing composition of the present invention. It is envisioned that the nicotine-containing composition can be beneficial in suppressing the symptoms associated with TS. See, Devor et al., The Lancet, Vol. 8670, p. 1046 (1989); Jarvik, British J. of Addiction, Vol. 86, pp. 571-575 (1991); McConville et al., Am. J. Psychiatry, Vol. 148 (6), pp. 793-794 (1991); Newhouse et al., Brit. J. Addic., Vol. 86, pp. 521-526 (1991); McConville et al., Biol. Psychiatry, Vol. 31, pp. 832-840 (1992); and Sanberg et al., Proceedings from Intl. Symp. Nic., S39 (1994).

### E. TREATMENT OF OTHER DISEASES OR DISORDERS

Another aspect to the present invention is a method for the prevention and treatment of other diseases or disorders, such as ulcerative colitis or obesity by administering a nicotine-containing composition to a subject susceptible to or suffering from such a disorder.

Inflammatory bowel disorders or diseases (IBD) encompass a spectrum of overlapping clinical diseases that appear to lack a common etiology. IBD, however, are characterized by chronic inflammation at various sites in the gastrointestinal (GI) tract. Illustrative IBD are regional enteritis (or Crohn's disease), idiopathic ulcerative colitis, idiopathic proctocolitis, pouchitis and infectious colitis. Symptoms of IBD may include persistent diarrhea, abdominal pain, fever, weight loss, joint pain, skin lesions and general fatigue. The inflammatory conditions of ulcerative colitis are confined to the colon, unlike Crohn's disease which can involve any portion of the intestinal tract.

Studies have suggested that an important epidemiolgic link exists between ulcerative colitis (UC) and a patient's smoking history. Several investigators have reported that the prevalence of UC in non-smokers is higher than in current smokers. Thus, a further embodiment of the present invention comprises administering to an individual suffering from UC the nicotine-containing composition of the present invention.

Yet further, it is envisioned that the nicotine-containing composition of the present invention can be used as a treatment for obesity or as a weight control therapy. It has been well established that smokers weight less than non-smokers. Intravenous nicotine infusion was shown to modestly increase the resting metabolic rate (6.5%) of smokers and non-smokers similarly. Also, in smokers and non-smokers alike, nasal nicotine solution insufflation significantly reduced the perceived taste intensity of dietary “fat”, but not “sweets”. From this, it appears that nicotine acts to decrease body weight through decreased calorie intake (i.e., appetite suppression) and increased metabolism. The mechanism for the observed appetite suppression is likely related to the increased serotonergic activity within the hypothalamus of the brain.

Thus, the present invention provides a therapeutic method to suppress appetite and/or prevent weight gain and/or induce weight loss in a subject in need of such therapy.

### F. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Preparation of Nicotine and Cocoa Powder Composition

A tablet, weighing around 400 mg, having the following composition (w/w): 1-6 mg nicotine (as base or salt, preferably hydrogen tartrate); diluent/filler, taste-masking, smoothening; and flavoring agent (cocoa powder around 50%); lipid ingredient (fatty components around 44%); buffering agent (sodium carbonate around 15 mg); sweetener (aspartame around 0.6%); and emulsifier/solubilizer (lecithin around 1%). Optional flavoring agent can also be included, for example, peppermint or vanilla flavor (0.5%). The nicotine may also be present in a complex, e.g., with a cation exchange resin or with cyclodextrin.

The composition is prepared as follows. Briefly, a part of the fatty components is melted. The solid components, i.e., nicotine, if in salt form, cocoa powder, aspartame, sodium carbonate and the optional flavoring agent if solid are added and mixed. A reduction of particle size of the solid components is performed by milling in a roll-refiner. If the solid components have already got the required particle size, e.g., by milling before the mixing with the fatty components, roll refining is dispensed with. After treatment in the roll-re-finer the mixture is mixed with the rest of the melted fatty components or remelted (if solidified) and mixed with the rest of the melted fatty components. A mixing of the melt is performed in a suitable mixer. The liquid components, i.e., lecithin, nicotine, if in the liquid base form, and the optional flavoring agent if liquid, are added. Tablets or other solid dosage forms are subsequently made using suitable techniques, such as molding, extrusion or congealing, including pastillation, when necessary after suitable preconditioning. Also other suitable manufacturing methods may be used.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

What is claimed is:

1. A nicotine-containing pharmaceutical composition comprising cocoa powder as a vehicle.

2. The nicotine-containing pharmaceutical composition of claim 1, wherein the cocoa powder comprises an amount that is at least a taste-masking effective amount of cocoa powder.

3. The nicotine-containing pharmaceutical composition of claim 1, wherein the cocoa powder is the diluent agent, filler agent, smoothening agent, and flavoring agent.

4. The nicotine-containing pharmaceutical composition of claim 1 further comprising at least one compound selected from the group consisting of sucrose, fructose, glucose, galactose, lactose, maltose, invert sugar, xylitol, sorbitol, maltitol, mannitol, isomalt, glycerol, polydextrose, and any mixture thereof.

5. The nicotine-containing pharmaceutical composition of claim 1 further comprising one or more lipid components.

6. The nicotine-containing pharmaceutical composition of claim 5, wherein the lipid is cocoa butter or cocoa butter alternatives.

7. The nicotine-containing pharmaceutical composition of claim 6, wherein the cocoa butter alternatives are selected from the group consisting of cocoa butter equivalents (CBE), cocoa butter substitutes (CBS), cocoa butter replacers (CBR) and cocoa butter improvers (CBI).

8. The nicotine-containing pharmaceutical composition of claim 5, wherein the lipid is selected from the group consisting of oils based on lauric and myristic acids, oils based on palmitic, oleic and stearic acids, oils based on oleic, linoleic and linolenic acids, and oils based on animal fat.

9. The nicotine-containing pharmaceutical composition of claim 8, wherein the oils based on lauric and myristic acids are coconut oil or palmkernel oil.

10. The nicotine-containing pharmaceutical composition of claim 8, wherein the oils based on palmitic, oleic and stearic acids are selected from the group consisting of palm oil, shea butter, karite butter, illipe butter, mango kernel oil, and sal fat.

11. The nicotine-containing pharmaceutical composition of claim 8, wherein the oils based on oleic, linoleic and linolenic acids are selected from the group consisting of corn oil, sunflower oil, hybrid sunflower oil, soybean oil, rapeseed oil, canola oil, olive oil, rice bran oil, cottonseed oil, peanut oil and groundnut oil.

12. The nicotine-containing pharmaceutical composition of claim 8, wherein the oils based on animal fat is fish oil, tallow, lard, or butterfat.

13. The nicotine-containing pharmaceutical composition of claim 5, wherein the lipid is a synthetic fat, reesterified fat, or hard fat.

14. The nicotine-containing pharmaceutical composition of claim of claim 5 further comprising a buffering agent, wherein the buffering agent is selected from the group consisting of sodium carbonates, sodium bicarbonates, sodium phosphates, sodium glycinates, sodium acetates, sodium gluconates, sodium glycerophosphates, potassium carbonates, potassium bicarbonates, potassium phosphates, potassium glycinates, potassium acetates, potassium gluconates, potassium glycerophosphates, ammonium carbonates, ammonium bicarbonates, ammonium phosphates, ammonium glycinates, ammonium acetates, ammonium gluconates, ammonium glycerophosphates and mixtures thereof

15. The nicotine-containing pharmaceutical composition of claim 14 further comprising at least one emulsifier/solubiliser.

16. The nicotine-containing pharmaceutical composition of claim 15, wherein the emulsifiers/solubilisers are selected from the group consisting of lecithin, a nonionic surfactant, an anionic surfactant, a zwitterionic surfactant and combinations with lecithin.

17. The nicotine-containing pharmaceutical composition of claim 16, wherein lecithin is soy lecithin or egg lecithin.

18. The nicotine-containing pharmaceutical composition of claim 16, wherein the nonionic surfactant is selected from the group consisting of poloxamer, polyoxyethylene alkyl ether, polyoxyethylene castor oil derivative, polyoxyethylene sorbitan fatty acid ester, monoglyceride, diglyceride, polyoxyethylene stearate, polyglycerolester of fatty acids and sorbitan fatty acid ester.

19. The nicotine-containing pharmaceutical composition of claim 16, wherein the anionic surfactant is selected from the group consisting of fatty acid, soap of fatty acid, lactylate, sodium lauryl sulfate and latanol.

20. The nicotine-containing pharmaceutical composition of claim 16, wherein the zwitterionic surfactant is a zwitterionic phospholipid.

21. The nicotine-containing pharmaceutical composition of claim 15 further comprising at least one sweetener, wherein the sweetener is selected from the group consisting of aspartame, acesulfame potassium, saccharine, cyclamate, glycyrrhizine, dihydrochalcones, stevisoide, thaumatin, monellin and neohesperidine.

22. The nicotine-containing pharmaceutical composition of claim 21 further comprising a flavoring agent, wherein the flavoring agent is selected from the group consisting of peppermint, coffee, orange and vanilla.

23. The nicotine-containing pharmaceutical composition of claim 22, wherein a unit dose comprises from about 0.5 mg to about 10 mg of nicotine, from about 17% to abaout 70% (w/w) of cocoa powder, from about 20% to about 50% (w/w) of a lipid component, from about 0.3% to about 3% (w/w) of a sweetener, from about 0% to about 10% (w/w) of a buffering agent, from about 0.3% to about 5% (w/w) of a emulsifier/solubilizer and from 0% to about 4% (w/w) of a flavoring agent.

24. The nicotine-containing pharmaceutical composition of claim 22, wherein the unit dose comprises from about 1 mg to about 6 mg of nicotine, 50% (w/w) cocoa powder, 44% (w/w) lipid components, 15 mg sodium carbonate, 0.6% (w/w) aspartame and/or acesulfame potassium and about 1% (w/w) lecithin.

25. The nicotine-containing pharmaceutical composition of claim 22, wherein the composition is formulated for oral administration.

26. A method for nicotine replacement therapy comprising the step of administering to a subject in need of such therapy a unit dose of a nicotine-containing pharmaceutical composition, wherein the unit dose of the composition comprises from about 0.5 mg to about 10 mg of nicotine, from about 17% to about 70% (w/w) cocoa powder, from about 20% to about 50% (w/w) a lipid component, from about 0% to about 10% (w/w) of a buffering agent, from about 0.3% to about 3% (w/w) of a sweetener and from about 0.3% to about 5% of an emulsifier/solubilizer.

27. The method of claim 26, wherein administering is via an oral route.

28. The method of claim 26 further comprising administering to said subject a second formulation of nicotine.

29. The method of claim 28, wherein the second formulation is administered via a device for transdermal administration of nicotine.

30. The method of claim 28, wherein the second formulation is administered nasally or buccally.

31. The method of claim 28, wherein the second formulation is administered via inhalation.

32. A method of treating a subject suffering from nicotine addiction comprising administering to said subject the composition of claim 23.

33. A method of treating a subject suffering from Alzheimer's disease comprising administering to said subject the composition of claim 23.

34. A method of treating a subject suffering from Parkinson's disease comprising administering to said subject the composition of claim 23.

35. A method of treating a subject suffering from Tourette's syndrome comprising administering to said subject the composition of claim 23.

36. A method of treating a subject suffering from ulcerative colitis comprising administering to said subject the composition of claim 23.

37. A method of treating a subject suffering from obesity comprising administering to said subject the composition of claim 23.

38. A method of controlling the weight of a subject comprising administering to said subject the composition of claim 23.
